(19) United States
(12) Patent Application Publication
(10) Pub. No.: US 2023/0293858 A1
(43) Pub. Date: Sep. 21, 2023

(54) GUIDEWIRE WITH PORTIONS OF REDUCED STIFFNESS

(71) Applicant: Boston Scientific Medical Device Limited, Galway (IE)

(72) Inventors: Christian Balkovec, Kitchener (CA); Brock Miller, Toronto (CA); Yun Uhm, North York (CA); Foss Jiao, Woodbridge (CA); Yan Yue, Mississauga (CA)

(21) Appl. No.: 18/184,794

(22) Filed: Mar. 16, 2023

Related U.S. Application Data
(60) Provisional application No. 63/320,383, filed on Mar. 16, 2022.

(51) Int. Cl. A61M 25/09

(52) U.S. Cl. CPC A61M 25/09; A61M 2025/09166; A61M 2025/09175

(57) ABSTRACT

A method and apparatus are disclosed for a guidewire that comprises an elongate member with a proximal and distal portion. The distal portion includes a distal curved portion, ending in a distal tip, and a tapered region that reduces the cross-section from a larger cross-section to a smaller cross-section. The elongate member further includes a plurality of flattened regions positioned at discrete locations along the elongate member. Each of the plurality of flattened regions are separated from one another by an unflattened region whereby when a force is exerted onto the guidewire, the guidewire undergoes bending at the plurality of flattened regions.

### CROSS-REFERENCE TO RELATED APPLICATIONS

This application claims the benefit of U.S. Provisional Application No. 63/320,383, entitled “A GUIDEWIRE WITH PORTIONS OF REDUCED STIFFNESS,” and filed Mar. 16, 2022, which is hereby incorporated by reference in its entirety.

### BACKGROUND

The disclosure relates to devices and methods for puncturing a septum of a heart. More specifically, the disclosure relates to a guidewire configured to perforate the intra-atrial septum.

Medical guidewires are typically constructed using a metal core mandrel with a round (circular) cross-section. In order to make the guidewire more compliant, and less traumatic, a distal portion of the core wire is ground down, tapering from a larger outer diameter to a smaller outer diameter. Additionally, the core mandrel may be shaped or curved to assume an atraumatic configuration, such as a J-shape, in order to mitigate tissue trauma. In this configuration, the contact force experienced by the guidewire upon tissue contact is spread over a larger surface area.

To retain the curve of the guidewire, the mandrel may be crushed or flattened. This results in the metal mandrel being work-hardened, thereby providing curve retention properties, and enhancing the atraumatic nature of the guidewire. However, when the mandrel is crushed or flattened, a bending point is created at the point where the cross-section changes from a round or circular cross-section to the crushed or flattened cross-section. In other words, at the intersection of the circular cross-section and the start of the flattened portion a bending point is formed or created when the guidewire is pressed against a tissue. Thus, if the guidewire is manipulated roughly or undergoes significant force against the tissue, the guidewire may become kinked or pinched (excessive bending resulting in plastic deformation) at the bending point. In some instances, if the guidewire is subjected to repeated kinking or pinching, the guidewire may break.

### SUMMARY

In one broad aspect of the disclosure, a guidewire comprises an elongate member with a proximal and distal portion. The distal portion includes a distal curved portion, ending in a distal tip, and a tapered region that reduces the cross-section from a larger cross-section to a smaller cross-section. The elongate member further includes a plurality of flattened regions positioned at discrete locations along the elongate member. Each of the plurality of flattened regions are separated from one another by an unflattened region whereby when a force is exerted onto the guidewire, the guidewire undergoes bending at the plurality of flattened regions.

### BRIEF DESCRIPTION OF THE DRAWINGS

In order that the invention may be readily understood, embodiments of the invention are illustrated by way of examples in the accompanying drawings, in which:

FIG. 1 depicts a side view of an embodiment of the prior art of a guidewire; and

FIG. 2 depicts a side view of the prior art of a guidewire comprising a curved distal portion; and

FIG. 3 depicts a side view of a guidewire comprising a plurality of reduced stiffness portions and a curved distal portion; and

FIG. 4 depicts a side perspective view of a guidewire depicted in FIG. 3; and

FIG. 5 depicts a front view of a guidewire comprising a radiopaque coil; and

FIG. 6 depicts a side view of a guidewire comprising a plurality of tapered portions; and

FIGS. 7A - 7B depicts a cross-section view of a guidewire of an alternative embodiment; and

FIGS. 8A - 8B depicts a side view of a guidewire with a plurality of coiled portions of an alternative embodiment; and

FIGS. 9A - 9B depicts a side view of a guidewire with a plurality of less stiff portions of an alternative embodiment.

### DETAILED DESCRIPTION

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As further described hereinbelow, the present invention provides a guidewire comprising a plurality of crushed or flattened portions to provide enhanced curve retention and reduce the likelihood of kinking or breakage.

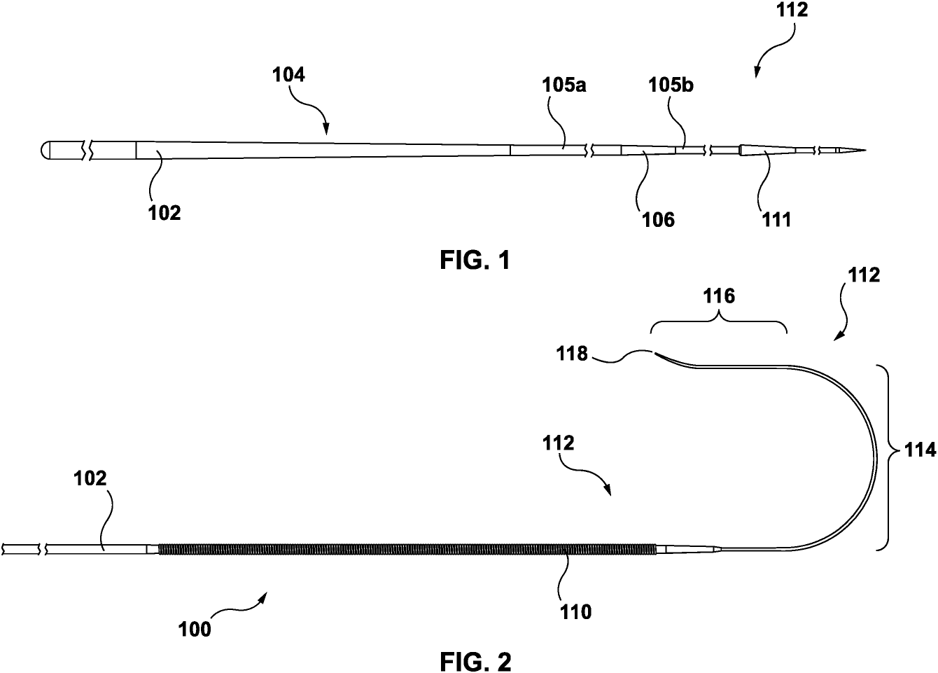

With reference now to FIG. 1 of the prior art depicts a core mandrel 102 of a guidewire that is configured for puncturing a target tissue (e.g., a septum of a heart). The mandrel 102 may comprise multiple tapered portions, separated by non-tapered regions. This configuration provides greater compliance over a shorter length of the mandrel. The tapered portions and intermediate regions may be formed by grinding down the mandrel 102. The core mandrel 102 may comprise a first, proximal, tapered region 104 and a second, intermediate, tapered region 106. The guidewire 100 may further comprise a radiopaque coil 110 (not shown) and coil stopper 111. Transition regions 105a and 105b may be positioned between the tapered regions 104, 106. The transition regions 105a and 105b are regions of constant diameter.

With reference to FIG. 2 of the prior art, the guidewire 100 comprises a curved distal region 114 configured to assume an atraumatic shape. The curved distal region 114 may comprise a straightened portion 116 positioned distal to the curve 114, thereby forming a J-shape, and terminating at the distal tip 118. The curved distal region 114 extends the length of the distal portion 112, with the distal portion 112 terminating at the distal tip 118. The distal tip 118 is configured to puncture the target tissue. The curved distal region 114 redirects the distal tip 118 such that it does not contact tissue during advancement, thereby preventing inadvertent damage to surrounding tissues.

To promote curve retention, the distal portion 112 may be crushed or flattened. In some embodiments, the flattened region extends along the length of the distal portion 112. In an alternative embodiment, the flattened region may be positioned at a different location along the guidewire 100 and does not necessarily need to extend the entire length of the distal portion 112. The point where the mandrel 102 changes from a circular cross-section to the flattened cross-section is the most compliant. This results in a bending point at that location when there is force exerted against the curved distal portion 112. Due to this, the guidewire 100 may kink or become pinched at this location which, in some instances, may result in breakage.

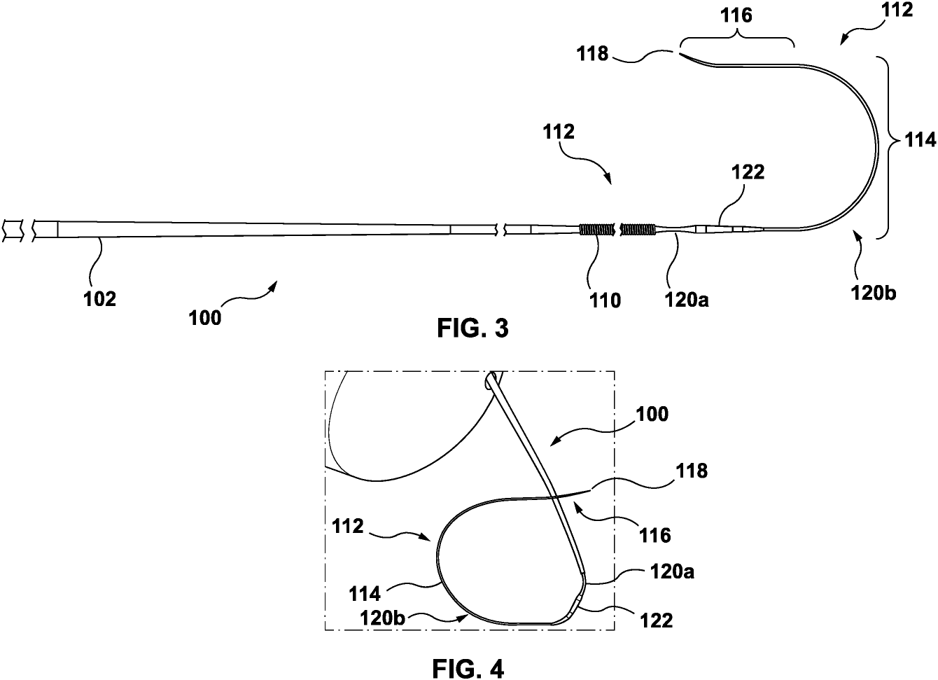

FIG. 3 depicts a side view of a guidewire according to one aspect of the present invention. Typically, guidewires 100 can vary in length between 80.0 cm (31.5″) to 450.0 cm (177.2″) and tapers down from a larger cross-section to a smaller cross-section. This results in the distal portion 112 having the smallest cross-section, and thus, being compliant and less traumatic when contacting tissues. The mandrel 102 may have a round or circular cross-section with an outer diameter between 0.355 mm (0.014″) to 0.965 mm (0.038″); alternatively, the cross-section of the mandrel may assume any other shape. The mandrel 102 may comprise multiple tapered portions, separated by non-tapered regions. This configuration provides greater compliance over a shorter length of the mandrel. The tapered portions and intermediate regions may be formed by grinding down the mandrel 102. The core mandrel 102 may comprise a first, proximal, tapered region 104 and a second, intermediate, tapered region 106. The guidewire 100 may further comprise a radiopaque coil 110 (not shown) and coil stopper 111. Transition regions 105a and 105b may be positioned between the tapered regions 104, 106 and the coil stopper 111. The transition regions 105a and 105b are regions of constant diameter. In one embodiment, the radiopaque coil 110 would be positioned overtop the transition region 105b proximal to the coil stopper 111. Alternatively, the mandrel 102 may have a smooth taper from a larger outer diameter to a smaller outer diameter.

The guidewire further comprises a curved distal region 114. The curved distal region 114 may comprise a straightened portion 116 positioned distal to the curve 114, thereby forming a J-shape, and terminating at the distal tip 118. In an alternative embodiment, the distal curve 114 may be in the form of a pigtail curve. The guidewire 100 may be configured such that when the guidewire 100 is constrained or positioned within an ancillary device, the distal portion 112 may assume a straightened configuration. When the guidewire 100 is unconstrained, it may assume the curved configuration. The curved distal region 114 extends the length of the distal portion 112, with the distal portion 112 terminating at the distal tip 118. The distal tip 118 may be a sharp tip which requires mechanical force to puncture the target tissue. The curved distal region 114 redirects the sharp distal tip 118 such that it does not contact tissue during advancement, thereby preventing inadvertent damage to surrounding tissues. The distal tip 118 may be configured to deliver energy to the tissue. In one example, the distal tip 118 may be an electrode that electrically communicates with an energy generator.

The mandrel 102 may comprise multiple tapered portions, separated by non-tapered regions. This configuration provides greater compliance over a shorter length of the mandrel. The tapered portions and intermediate regions may be formed by grinding down the mandrel 102. The core mandrel 102 may comprise a first, proximal, tapered region 104 and a second, intermediate, tapered region 106. The guidewire 100 may further comprise a radiopaque coil 110 (not shown) and coil stopper 111. In one embodiment, the guidewire 100 may comprise a plurality of flattened portions 120a and 120b. The flattened portions 120a and 120b have, comparatively, lower stiffness than the unflattened mandrel 102. The flattened portions 120a and 120b may be positioned along the guidewire 100 where the majority of stresses during use are expected to occur. Typically, these are near the distal portion 112 that may contact tissues directly. The flattened portions 120a and 120b may be positioned such that there is an unflattened portion 122 (e.g., a portion of mandrel 102 with a circular cross-section) within relative proximity to each other. In a particular embodiment, the flattened portions 120a and 120b may be positioned such that there is an unflattened portion 122 separating them. In other words, the flattened portions 120a and 120b may alternate with unflattened portions 122 along the length of the guidewire 100. With this configuration, the guidewire 100 distributes bending across multiple points. In other words, as the guidewire 100 contacts tissue, the guidewire 100 undergoes bending at the plurality of flattened portions 120a and 120b (as depicted in FIG. 4). Although a guidewire 100 with two flattened portions is described, it would be appreciated that any number of flattened portions may be positioned at various positions along the guidewire 100. These flattened portions may be positioned such that they alternate in between unflattened portions 122.

In an alternative embodiment, the unflattened portions 122 may undergo some flattening but to a lesser extent than the flattened portions 120a and 120b. This would still result in the flattened portions 120a and 120b to undergo bending while the less flattened portions would be stiffer than the flattened portions 120a and 120b.

The distribution of bending along the guidewire 100 reduces the potential of the metal breaking through the Bauschinger effect. When metal is work-hardened in the direction it is bent, it strengthens the metal in that direction while reducing the strength (weakening it) in the reverse direction. Continuous strain reversals via bending at the same location on the guidewire 100 will eventually lead to failure of the material. In some instances, the device may withstand multiple strain reversals (repeated back-and-forth bending at a single point) prior to breaking; in other instances, the device may only withstand a single strain reversal. The distribution of bending across multiple points reduces the likelihood of kinking and plastic deformation. Without plastic deformation, the Bauschinger effect won’t occur. Additionally, configuring the guidewire 100 with alternating flattened 120a and 120b and unflattened 122 portions enables the guidewire 100 to twist more easily by facilitating flexural-torsional buckling of the unflattened portion as it seeks a lower-stress state, therefore reducing the likelihood of reoccurring kinking at the same location on the guidewire 100.

The flattened portions, in some embodiment, are at least 0.3 mm in length, or in other embodiment, at least 1.0 mm in length to allow for a smooth transition from the unflattened height to the flattened height. Ideally, the flattened height of the mandrel 102 may be flattened to a height such that the guidewire 100 selectively bends at the flattened portions. In some embodiments, the first (proximal) flattened portion 120a may have a length between 1.5 mm (0.059″) to 2.0 mm (0.079″). The second (distal) flattened portion 120b may have a length between 20.0 mm (0.797″) to 22.0 mm (0.866″) and a height of 0.089 mm (0.0035″) to 0.114 mm (0.0045″). In yet another embodiment, a round or unflattened portion 122 may be positioned between the flattened portions 120a and 120b. The unflattened portion 122, in embodiments, can be at least 0.3 mm in length, or at least 1.0 mm in length, in other embodiments, to ensure that each flattened portion 120a and 120b can be able to bend independent from each other, thereby preventing pinching at one single region. The unflattened portion 122, in some embodiments, has a greater stiffness than each flattened portion 120a and 120b, in a least one plane of reference, to help ensure bending selectively occurs at the flattened portions. Furthermore, to prevent the need for additional reinforcing components (e.g., a coil), the outer diameter of the mandrel 102 undergoing flattening can be no less than 0.152 mm (0.006″).

In another embodiment, the mandrel 102 may be composed of nitinol. Nitinol possesses super elastic properties, allowing the guidewire 100 to be kink-resistant, removing the need for additional reinforcing components (e.g., inner and/or outer coils over the flattened region(s)). In an alternative embodiment, the core mandrel 102 may be composed of stainless steel. However, stainless steel may be more prone to kinking and, thus, reinforcing components such as an inner or outer coil over the flattened portions may be necessary.

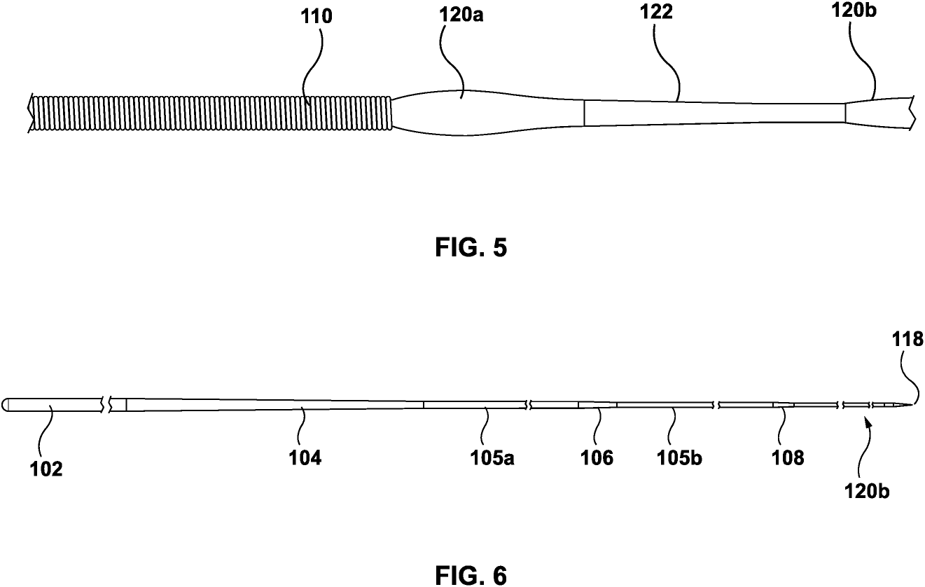

In some embodiments, the guidewire 100 may comprise visualization elements to enable the device to be visible under various visualization systems (e.g., fluoroscopy and/or ultrasound). In one embodiment, the guidewire 100 may include a radiopaque coil 110 (as depicted in FIG. 2 and FIG. 3). The radiopaque coil 110 may comprise a round wire that is coiled and close wound around the diameter of the mandrel 102. The radiopaque coil 110 may be positioned proximal the plurality of flattened portions 120a and 120b. In some embodiments, the portion of the mandrel 102 comprising the radiopaque coil 110 may be ground down such that when the radiopaque coil 110 is positioned over the mandrel 102, it does not significantly increase the outer diameter of the device. The radiopaque coil 110 may be composed of any material that may be visible under fluoroscopy, such as platinum-iridium, tungsten, gold, or any other material which attenuates x-rays. The radiopaque coil 110 may be held in place distally by a coil stopper 111. In some embodiments, a flattened region 120a may act as the stopper 111, where the radiopaque coil 110 may be positioned proximal to the flattened region 120a. In this embodiment, the flattened region 120a may be flattened to a specific width to hold the coil 110 in place (as seen in FIG. 5), for example, to a width of 0.432 mm (0.017″) to 0.609 mm (0.024″). In an alternative embodiment, the coil 110 may be placed between two of the plurality of flattened portions 120a and 120b, where each of the flattened portions 120a and 120b act as a stopper 111. Alternatively, a radiopaque marker such as a band or ring of dense metal contained on the guidewire 100 via a stopper 111 may be used. Further, a radiopaque coating that is sprayed or deposited directly on the guidewire 100 and adheres to the guidewire 100 surface without use of any stoppers 111 may be used instead of a radiopaque coil 110.

In a specific example, with reference to FIG. 6, a guidewire 100 is disclosed, with a distal tip 118, configured for puncturing a target tissue. The guidewire 100 comprises a mandrel 102 composed of super elastic nitinol with a circular or round cross-section. The length of the guidewire 100 may be between 180 cm (70.8″) to 230 cm (90.5″) with an outer maximum diameter ranging between 0.787 mm (0.031″) to 0.80 mm (0.0315″). The start of the proximal tapered region 104 may be positioned 76.61 mm (3.01″) to 79.15 mm (3.11″) from the distal tip 118. The proximal tapered region 104 length may range from 15.75 mm (0.620″) to 22.35 mm (0.880″) and tapers from the maximum outer diameter (0.787 mm (0.031″) to 0.80 mm (0.0315″)) to an outer diameter of between 0.399 mm (0.0157″) to 0.414 mm (0.0163″). The start of the middle tapered region 106 may be positioned 43.84 mm (1.72″) to 44.6 mm (1.75″) from the distal tip 118. The middle tapered portion 106 length may range from 2.29 mm (0.09″) to 2.79 mm (0.11″) in length and reduce the outer diameter such that it ranges between 0.297 mm (0.0117″) to 0.312 mm (0.0123″). The start of the distal tapered region 108 may be positioned 20.65 mm (0.81″) to 21.42 mm (0.84″) from the distal tip 118. The length of the distal tapered region 108 may range between 1.02 mm (0.04″) to 1.52 mm (2.04″). The outer diameter tapers down until it reaches between the range of 0.1955 mm (0.0077″) to 0.2108 mm (0.0083″). In between each tapered region 104, 106, and 108 is a transition region 105a and 105c.

In this example, the guidewire 100 further comprises two flattened portions 120a and 120b. The first, proximal, flattened portion 120a may extend for a length between 1.5 mm (0.059″) to 2.0 mm (0.079″) and a width of 0.432 mm (0.017″) to 0.609 mm (0.024). The location of the first, proximal, flattened portion 120a is at the location of the second transition region 105b, between the middle tapered region 106 and the distal tapered region 108. The second, distal, flattened portion extends to the distal tip 118 and may range between 20.0 mm (0.797″) to 22.0 mm (0.866″) and a height of 0.089 mm (0.0035″) to 0.114 mm (0.0045″). An unflattened, round, section 122 of guidewire 100 is situated between the proximal 120a and distal 120b flattened portions. The length of the unflattened, round, section 122, may be between 1 mm and 3.0 mm. The distal tip 118 of the guidewire 100 comprises a sharp tip with a radius less than or equal to 0.001″ for mechanically puncturing the target tissue.

The guidewire 100 further comprises a J-shaped curved distal region 114. The curved distal region 114 extends the entire length of the distal tapered region 108. The curve may have a diameter that ranges between 7.0 mm (0.27″) to 9.0 mm (0.35″). Distal to the curve, the straightened region 116 has a length between 4.5 mm (0.177″) to 6.5 mm (0.255″), terminating at the distal tip 118.

A radiopaque coil 110 is positioned on the guidewire 100 to allow users to visualize the device under fluoroscopy and/or ultrasound. The radiopaque coil 110 is composed of platinum-iridium and positioned proximal to the first, proximal, flattened portion 120a and may comprise a length between 20.0 mm (0.797″) to 22.0 mm (0.866″). In this example, the radiopaque coil 110 may have an inner diameter between 0.317 mm (0.0125″) and 0.342 mm (0.0135″) and an outer diameter between 0.419 mm (0.0165″) and 0.445 mm (0.0175″).

Although a plurality of flattened portions 120a and 120b has been described above, the flattened portions 120a and 120b can be portions of reduced stiffness, thereby being more compliant, and may comprise various modifications to achieve a portion of reduced stiffness.

In one alternative embodiment, the plurality of portions with reduced stiffness may comprise a plurality of portions with a reduction in outer diameter. These portions would have a diameter that is reduced compared to the areas directly adjacent to it, resulting in bending at these areas.

In a further alternative embodiment, the portions with reduced stiffness may be achieved by introducing surface irregularities to the mandrel 102. As an example, the mandrel 102 may comprise portions with cut-outs or slits which will reduce the stiffness of those portions, compared to the adjacent portions that do not comprise any surface irregularities, which will result in those portions undergoing bending.

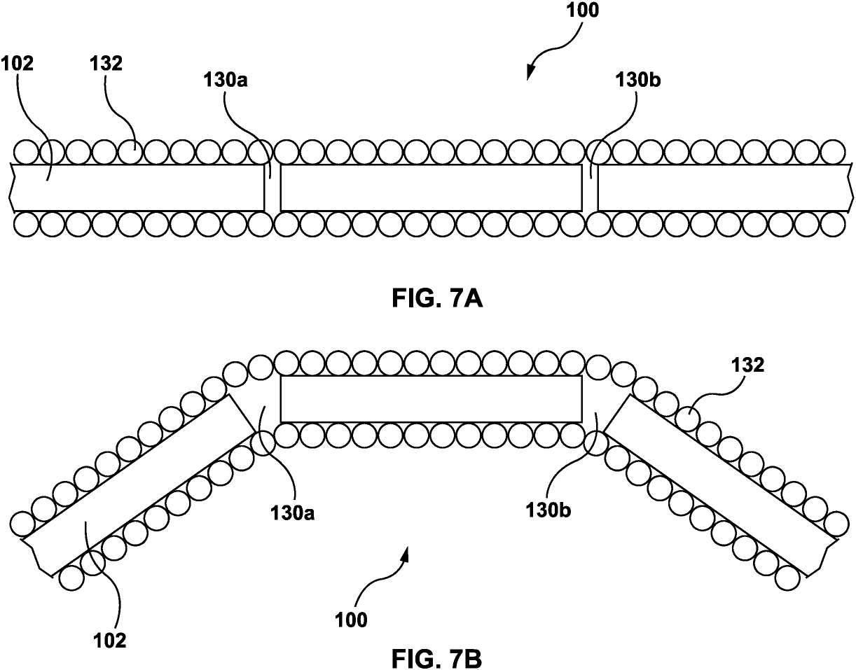

An alternative embodiment is depicted in FIGS. 7a - 7b. In this embodiment, the mandrel 102 may be discontinuous or broken along its length 130a and 130b. To hold the discontinuous mandrel 102 together, an outer coil 132 may be wound around the mandrel 106, resulting in a single device. In some embodiments, the outer coil 132 may be composed of radiopaque material (such as platinum-iridium) to enable visualization under fluoroscopy. As shown in FIG. 7b, the breaks or discontinuous portions 130a and 130b would bend when force is applied to the guidewire 100.

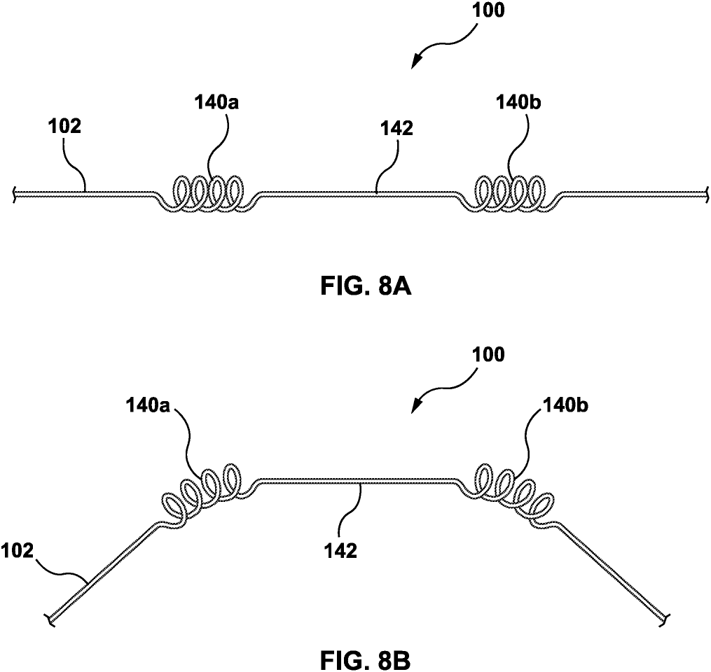

Another alternative embodiment may comprise a mandrel 102 with a plurality of coiled portions 140a and 140b, as illustrated in FIGS. 8a - 8b. The coiled portions 140a and 140b of the mandrel 102 act as flexible spring section. The coiled portions 140a and 140b may be separated by a straight segment 142. As depicted in FIG. 7b, the coiled portions 140a and 140b would deflect and bend when force is applied to the guidewire 100.

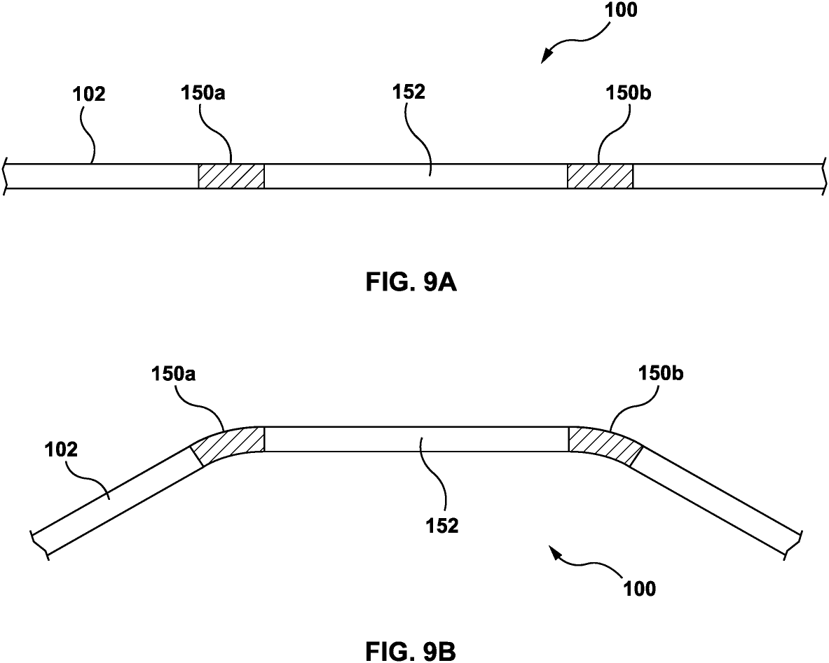

In another alternative embodiment, the guidewire 100 may comprise a mandrel 102 composed of various materials. For example, as illustrated in FIGS. 9a - 9b, the mandrel 102 may be formed with portions of material that have lower stiffness 150a and 150b. separated by material with higher stiffness 152. In some embodiments the material of higher stiffness 152 may be a different material than the mandrel 102. In alternative embodiments, the material of higher stiffness 152 may be the same material as the mandrel 102. The stiffer and less stiff materials may be secured (e.g., welded) together, forming a single device. Similar to previous embodiments, the portions of the guidewire 100 comprising the material of lower stiffness 150a and 150b would selectively bend (as seen in FIG. 9b).

It is appreciated that these means of introducing reduced stiffness to the mandrel 102 of the guidewire 100 may be used independently or in combination with one another. Additionally, a person skilled in the art would recognize that each of the plurality of reduced stiffness portions may vary between one another. For example, the first portion of reduced stiffness may be less than the second portion of reduced stiffness with both the first and second portions of reduced stiffness being less than the stiffness of the sections adjacent to them.

Various exemplary embodiments are listed below:

1) A guidewire comprising:
an elongate member comprising:
   a proximal portion;
a distal portion, the distal portion comprising:
   a distal curved portion ending in a distal tip;
a tapered region, wherein the tapered region reduces a cross-section of the elongate member from a large cross-section to a small cross-section;
a plurality of flattened regions positioned at discrete locations along the elongate member; and,
wherein each of the plurality of flattened regions are separated from one another by an unflattened region;
whereby, when a force is exerted onto the guidewire, the guidewire undergoes bending at the plurality of flattened regions.

2) The guidewire of example 1, wherein the elongate member is composed of nitinol.

3) The guidewire of any one of examples 1 or 2, wherein the elongate member comprises a circular cross section.

4) The guidewire of any one of examples 1 to 3, further comprising a radiopaque coil positioned along the distal portion.

5) The guidewire of example 4, wherein the radiopaque coil is composed of platinum-iridium.

6) The guidewire of any one of examples 4 or 5, wherein the radiopaque coil is held in place by a coil stopper.

7) The guidewire of any one of examples 1 to 6, wherein the tapered region comprises a plurality of tapers, wherein the plurality of tapers are separated by an intermediate portion of constant diameter.

8) The guidewire of any one of examples 1 to 7, wherein the plurality of flattened regions comprises a proximal flattened region and a distal flattened region.

9) The guidewire of example 8, wherein the distal flattened region extends from the sharp distal tip and ends proximal to the curved distal portion.

10) The guidewire of any one of examples 1 to 9, wherein the curved distal portion is J-shaped.

11) The guidewire of any one of examples 1 to 9, wherein the curved distal portion is in a pigtail configuration.

12) The guidewire of any one of examples 1 to 11, wherein the distal tip comprises a sharp tip configured to puncture a target tissue.

13) The guidewire of any one of examples 1 to 11, wherein the distal tip comprises an electrode configured to deliver energy to a target tissue.

14) A guidewire comprising:
an elongate member having a first stiffness, the elongate member comprising:
   a proximal portion;
   a distal portion, the distal portion ending in a distal tip; and,
a plurality of regions of a second stiffness positioned at discrete locations along the elongate member and separated by a portion of the first stiffness;
wherein the second stiffness is less than that of the first stiffness;
whereby, when a force is exerted onto the guidewire, the guidewire undergoes bending at the plurality of regions of the second stiffness preventing kinking of the guidewire.

15) The guidewire of example 14, wherein the distal portion comprises a distal curved portion, ending at the distal tip.

16) The guidewire of any one of examples 14 or 15, wherein the distal curved portion is J-shaped.

17) The guidewire of any one of examples 14 or 15, wherein the distal curved portion is in a pigtail configuration.

18) The guidewire of any one of examples 14 to 17, wherein the distal tip is a sharp tip configured to puncture a target tissue.

19) The guidewire of any one of examples 14 to 18, wherein the distal tip is an electrode configured to deliver energy to a target tissue.

20) The guidewire of any one of examples 14 to 19, further comprising a radiopaque coil.

21) The guidewire of example 20, wherein the radiopaque coil is positioned along the distal portion.

22) The guidewire of any one of examples 19 or 21, wherein the radiopaque coil is composed of platinum-iridium.

23) The guidewire of any one of examples 19 to 22, wherein the radiopaque coil is held in place by a stopper.

24) The guidewire of any one of examples 14 to 23, wherein the elongate member is composed of nitinol.

25) The guidewire of any one of examples 14 to 24, wherein the distal portion comprises a tapered region, the tapered region tapering from a large cross-section to a small cross-section.

26) The guidewire of example 24, wherein the tapered region comprises a plurality of tapered portions and wherein each of the plurality of tapered portions are separated from one another by an intermediate portion having a constant cross-section.

27) The guidewire of example 26, wherein the plurality of tapered portions comprises a proximal tapered portion, a middle tapered portion, and a distal tapered portion.

28) The guidewire of any one of examples 1 to 27, wherein each of the plurality of regions of the second stiffness vary in stiffness from one another.

29) The guidewire of any one of examples 1 to 28, wherein the plurality of regions of the second stiffness comprises a plurality of flattened portions and wherein the portion of the first stiffness comprises an unflattened portion.

30) The guidewire of example 29, wherein the plurality of flattened portions comprises a proximal flattened portion and a distal flattened portion.

31) The guidewire of example 30, wherein the distal flattened portion extends from the distal tip and ends proximal to the curved distal region.

32) The guidewire of any one of examples 1 to 28, wherein the portion of the first stiffness comprises a first material and wherein the plurality of regions of the second stiffness comprises a plurality of regions composed of a second material.

33) The guidewire of example 32, wherein the first material is the same material as the elongate member.

34) The guidewire of any one of examples 1 to 28, wherein the plurality of regions of the second stiffness comprises a plurality of coiled regions and where the portion of the first stiffness comprises a straight portion.

35) The guidewire of any one of examples 1 to 28, wherein the plurality of regions of the second stiffness comprises a plurality of regions with surface irregularities.

36) The guidewire of example 35, wherein the surface irregularities comprises a series of cut-outs.

37) The guidewire of any one of examples 1 to 28, wherein the plurality of regions of the second stiffness comprises a plurality of regions selected from a group consisting of flattened portions, or less stiff material, or coiled portions, or a combination thereof.

38) The guidewire of any one of examples 1 to 37, wherein the elongate member comprises a circular cross-section.

39) A guidewire comprising:
an elongate member comprising:
   a proximal portion;
   a distal portion, the distal portion comprising:
      a distal curved portion ending in a distal tip;
a plurality of broken portions; and,
an outer coil, wherein the outer coil wraps around the elongate member such that the elongate member forms a single piece;
whereby, when a force is exerted onto the guidewire, the guidewire undergoes bending at the plurality of broken portions, preventing kinking of the guidewire.

The embodiment(s) of the invention described above is(are) intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A guidewire comprising:
an elongate member comprising:
a proximal portion;
a distal portion, the distal portion comprising:
a distal curved portion ending in a distal tip;
a tapered region, wherein the tapered region reduces a cross-section of the elongate member from a large cross-section to a small cross-section;
a plurality of flattened regions positioned at discrete locations along the elongate member; and,
wherein each of the plurality of flattened regions are separated from one another by an unflattened region;
whereby, when a force is exerted onto the guidewire, the guidewire undergoes bending at the plurality of flattened regions.

2. The guidewire of claim 1, wherein the elongate member is composed of nitinol.

3. The guidewire of claim 1, wherein the elongate member comprises a circular cross section.

4. The guidewire of claim 1, further comprising a radiopaque coil positioned along the distal portion.

5. The guidewire of claim 4 wherein the radiopaque coil is held in place by a coil stopper.

6. The guidewire of claim 1, wherein the tapered region comprises a plurality of tapers, wherein the plurality of tapers are separated by at least one intermediate portion of constant diameter.

7. The guidewire of of claim 6, wherein the plurality of flattened regions comprises a proximal flattened region and a distal flattened region.

8) The guidewire of claim 7, wherein the distal flattened region extends from the sharp distal tip and ends proximal to the curved distal portion.

9. The guidewire of claim 1, wherein the curved distal portion is J-shaped.

10. The guidewire of claim 1, wherein the curved distal portion is in a pigtail configuration.

11. The guidewire of claim 1, wherein the distal tip comprises an electrode configured to deliver energy to a target tissue.

12. A guidewire comprising:
an elongate member having a first stiffness, the elongate member comprising:
a proximal portion;
a distal portion, the distal portion ending in a distal tip; and,
a plurality of regions of a second stiffness positioned at discrete locations along the elongate member and separated by a portion of the first stiffness;
wherein the second stiffness is less than that of the first stiffness;
whereby, when a force is exerted onto the guidewire, the guidewire undergoes bending at the plurality of regions of the second stiffness preventing kinking of the guidewire.

13. The guidewire of claim 12, wherein the distal portion comprises a distal curved portion, ending at the distal tip.

14. The guidewire of claim 12, wherein the distal portion comprises a tapered region, the tapered region tapering from a large cross-section to a small cross-section.

15. The guidewire of claim 14, wherein the tapered region comprises a plurality of tapered portions and wherein each of the plurality of tapered portions are separated from one another by an intermediate portion having a constant cross-section.

16. The guidewire of claim 15, wherein the plurality of tapered portions comprises a proximal tapered portion, a middle tapered portion, and a distal tapered portion.

17. The guidewire of claim 12, wherein the plurality of regions of the second stiffness comprises a plurality of flattened portions and wherein the portion of the first stiffness comprises an unflattened portion.

18. The guidewire of claim 17, wherein the plurality of flattened portions comprises a proximal flattened portion and a distal flattened portion.

19. The guidewire of claim 18, wherein the distal flattened portion extends from the distal tip and ends proximal to the curved distal region.

20. A guidewire comprising:
an elongate member comprising:
a proximal portion;
a distal portion, the distal portion comprising:
a distal curved portion ending in a distal tip;
a plurality of broken portions; and,
an outer coil, wherein the outer coil wraps around the elongate member such that the elongate member forms a single piece;
whereby, when a force is exerted onto the guidewire, the guidewire undergoes bending at the plurality of broken portions, preventing kinking of the guidewire.
